(19) United States
(12) Patent Application Publication
(10) Pub. No.: US 2025/0127380 A1
(43) Pub. Date: Apr. 24, 2025

(54) ENDOSCOPE DEVICE AND REPLACEABLE INSERTION TUBE MECHANISM AND CATHETER THEREFOR

(71) Applicant: Chang-Bih SHIE, Tainan City (TW)

(72) Inventor: Chang-Bih SHIE, Tainan City (TW)

(21) Appl. No.: 18/921,452

(22) Filed: Oct. 21, 2024

(30) Foreign Application Priority Data
Oct. 24, 2023 (TW) 112140508

(51) Int. Cl. A61B 1/00; A61B 1/05; A61B 90/00

(52) U.S. Cl. CPC A61B 1/00068; A61B 1/00105; A61B 1/00114; A61B 1/00119; A61B 1/00124; A61B 1/00128; A61B 1/0014; A61B 1/053; A61B 90/03; A61B 2090/034

(57) ABSTRACT

An endoscope device for connection with a catheter having a connecting portion and a tubular body portion includes a handgrip mechanism and a replaceable insertion tube mechanism. The handgrip mechanism includes a handgrip unit, a connecting tube having a first mating end portion, a locking ring, and a signal connector signally connected to the handgrip unit. The replaceable insertion tube mechanism includes an insertion tube having opposite second mating end portion and insertion end portion, and an image capturing unit having an image capture device mounted on the insertion end portion and a signal terminal signally connected to the image capture device. The second mating end portion is detachably and coaxially connected to the first mating end portion. The locking ring is detachably and threadedly connected to the second mating end portion so as to secure the first and second mating end portions in an end-to-end manner.

### CROSS-REFERENCE TO RELATED APPLICATION

This application claims priority to Taiwanese Invention patent application No. 112140508, filed on Oct. 24, 2023, and incorporated by reference herein in its entirety.

### FIELD

The present disclosure relates to an endoscope device, more particularly to an endoscope device having an insertion tube mechanism that is replaceable.

### BACKGROUND

When performing an insertion operation of a catheter, such as a nasogastric tube or a feeding tube, because the tubular body of this type of catheter is too soft, a steel wire is pre-inserted into the catheter during manufacture for increasing a supporting force thereof. During an insertion process of the catheter, an endoscope is also inserted side by side with the catheter to provide visual guidance. However, this method of inserting the catheter and an insertion tube of the endoscope side by side will cause discomfort to a patient during the insertion process because the structure of the two tubular bodies becomes thicker, and may even squeeze and scratch the mucosa of the body cavity of the patient.

Since the diameter of the insertion tube of an existing endoscope device is fixed so that the insertion tube cannot be replaced, when a catheter is to be inserted into the body cavity of a patient, it is necessary to select an endoscope device having an insertion tube with a suitable diameter according to the body shape of the patient, and the medical institution must prepare endoscope devices of various insertion tube sizes. As such, the equipment and installation costs of the medical institution will be significantly increased.

In addition, to perform an insertion operation of a urinary catheter, in order to push forward the urinary catheter through the prostate, a tube material that is hard and not easy to bend and deform is used for the urinary catheter. Because currently blind insertion is performed without knowing the extent of the patient's prostate enlargement, it often leads to frequent impact of the urinary catheter's front end against the prostate, causing intense pain and discomfort to the patient. When the urinary catheter cannot pass forward through the prostate section, the medical personnel will pull out the urinary catheter and then try other urinary catheters with smaller diameters one by one until an indwelling operation of the urinary catheter is successfully completed. This method of blindly inserting the urinary catheter is rather unfriendly to the patient and can easily result in waste of resources.

### SUMMARY

Therefore, an object of the present disclosure is to provide an endoscope device that can alleviate at least one of the drawbacks of the prior art.

Accordingly, the endoscope device is suitable for connection with a catheter. The catheter includes a connecting portion and a tubular body portion. The endoscope device includes a handgrip mechanism and a replaceable insertion tube mechanism. The handgrip mechanism includes a handgrip unit, a connecting unit connected to the handgrip unit and having a first mating end portion distal to the handgrip unit, and a signal connector signally connected to the handgrip unit and exposed from the first mating end portion. The connecting unit includes a connecting tube having the first mating end portion, and a first locking ring sleeved over the first mating end portion and movable along a length of the connecting tube.

The replaceable insertion tube mechanism includes an insertion tube unit having a second mating end portion and an insertion end portion opposite to each other along a length thereof, and an image capturing unit mounted on the insertion tube unit. The insertion tube unit includes an insertion tube having the second mating end portion and the insertion end portion and being configured to be insertable through the insertion end portion from the connecting portion to the tubular body portion of the catheter. The image capturing unit has an image capture device mounted on the insertion end portion, and a signal terminal signally connected to the image capture device and exposed from the second mating end portion. The second mating end portion is detachably and coaxially connected to the first mating end portion, and brings the signal terminal to detachably and signally connect with the signal connector. The image capture device is configured to capture image outside an end of the tubular body portion of the catheter. The first locking ring is detachably and threadedly connected to the second mating end portion so as to secure the first and second mating end portions in an end-to-end manner. The first locking ring has a first engaging ring portion sleeved threadedly on the second mating end portion, and a second engaging ring portion configured to sleeve threadedly on the connecting portion of the catheter.

Another object of this disclosure is to provide an endoscope device that can alleviate at least one of the drawbacks of the prior art.

Accordingly, the endoscope device is suitable for connection with a catheter. The catheter includes a connecting portion and a tubular body portion. The endoscope device includes a handgrip mechanism and a replaceable insertion tube mechanism. The handgrip mechanism includes a handgrip unit, a connecting unit connected to the handgrip unit and having a first mating end portion distal to the handgrip unit, and a signal connector signally connected to the handgrip unit and exposed from the first mating end portion. The connecting unit includes a connecting tube having the first mating end portion.

The replaceable insertion tube mechanism includes an insertion tube unit, and an image capturing unit mounted on the insertion tube unit. The insertion tube unit includes an insertion tube having a second mating end portion and an insertion end portion opposite to each other along a length thereof, and a third locking ring sleeved on the second mating end portion and movable along the length of the insertion tube. The image capturing unit has an image capture device mounted on the insertion end portion, and a signal terminal signally connected to the image capture device and exposed from the second mating end portion. The second mating end portion is detachably and coaxially connected to the first mating end portion, and brings the signal terminal to detachably and signally connect with the signal connector. The insertion tube is configured to be insertable through the insertion end portion from the connecting portion to the tubular body portion of the catheter. The image capture device is configured to capture image outside an end of the tubular body portion of the catheter. The third locking ring has a limiting ring portion sleeved on the second mating end portion and movable toward the insertion end portion, and a third engaging ring portion configured to sleeve threadedly on the connecting portion of the catheter.

A yet another object of this disclosure is to provide an endoscope device that can alleviate at least one of the drawbacks of the prior art.

Accordingly, the endoscope device is suitable for connection with a catheter. The catheter includes a connecting portion and a tubular body portion. The endoscope device includes a handgrip mechanism and a replaceable insertion tube mechanism. The handgrip mechanism includes a handgrip unit, a connecting unit connected to the handgrip unit and having a first mating end portion distal to the handgrip unit, and a signal connector signally connected to the handgrip unit and exposed from the first mating end portion. The connecting unit includes a connecting tube having the first mating end portion, and a first locking ring sleeved over the first mating end portion and movable along a length of the connecting tube.

The replaceable insertion tube mechanism includes an insertion tube unit having a second mating end portion and an insertion end portion opposite to each other along a length thereof, and an image capturing unit mounted on the insertion tube unit. The image capturing unit has an image capture device mounted on the insertion end portion, and a signal terminal signally connected to the image capture device and exposed from the second mating end portion. The image capture device is configured to capture image outside an end of the tubular body portion of the catheter. The insertion tube unit includes an insertion tube having the second mating end portion and the insertion end portion, and a third locking ring sleeved on the second mating end portion and movable along a length of the insertion tube.

The second mating end portion is detachably and coaxially connected to the first mating end portion, and brings the signal terminal to detachably and signally connect with the signal connector. The first locking ring is detachably and threadedly connected to the second mating end portion so as to secure the first and second mating end portions in an end-to-end manner. The insertion tube is configured to be insertable through the insertion end portion from the connecting portion to the tubular body portion of the catheter. The third locking ring has a limiting ring portion sleeved on the second mating end portion and movable toward the insertion end portion, and a third engaging ring portion configured to sleeve threadedly on connecting portion of the catheter.

A still another object of this disclosure is to provide an endoscope device that can alleviate at least one of the drawbacks of the prior art.

Accordingly, the endoscope device is suitable for connection with a catheter. The catheter includes a connecting portion and a tubular body portion. The endoscope device includes a handgrip mechanism and a replaceable insertion tube mechanism. The handgrip mechanism includes a handgrip unit, a connecting unit connected to the handgrip unit and having a first mating end portion distal to the handgrip unit, and a signal connector signally connected to the handgrip unit and exposed from the first mating end portion. The connecting unit includes a connecting tube having the first mating end portion.

The replaceable insertion tube mechanism includes an insertion tube unit having a second mating end portion and an insertion end portion opposite to each other along a length thereof, and an image capturing unit mounted on the insertion tube unit. The image capturing unit has an image capture device mounted on the insertion end portion, and a signal terminal signally connected to the image capture device and exposed from the second mating end portion. The image capture device is configured to capture image outside an end of the tubular body portion of the catheter. The insertion tube unit includes an insertion tube having the second mating end portion and the insertion end portion, a second locking ring sleeved over the second mating end portion and movable along a length of the insertion tube, and a third locking ring sleeved on the second mating end portion and movable along the length of the insertion tube. The second mating end portion is detachably and coaxially connected to the first mating end portion, and brings the signal terminal to detachably and signally connect with the signal connector. The second locking ring is detachably and threadedly connected to the first mating end portion so as to secure the first and second mating end portions in an end-to-end manner. The insertion tube is configured to be insertable through the insertion end portion from the connecting portion to the tubular body portion of the catheter. The third locking ring has a limiting ring portion sleeved on the second mating end portion and movable toward the insertion end portion, and a third engaging ring portion configured to sleeve threadedly on the connecting portion of the catheter.

A further object of this disclosure is to provide a replaceable insertion tube mechanism that can alleviate at least one of the drawbacks of the prior art.

Accordingly, the replaceable insertion tube mechanism is suitable for mounting on a handgrip mechanism of an endoscope device, and is suitable for a catheter to sleeve thereon. The catheter includes a connecting portion and a tubular body portion. The replaceable insertion tube mechanism includes an insertion tube unit and an image capturing unit. The insertion tube unit includes an insertion tube having a second mating end portion and an insertion end portion opposite to each other along a length thereof, and a third locking ring sleeved on the second mating end portion and movable along the length of the insertion tube.

The second mating end portion is configured to detachably and coaxially connect with the handgrip mechanism. The insertion tube is configured to be insertable through the insertion end portion from the connecting portion to the tubular body portion of the catheter. The third locking ring has a limiting ring portion sleeved on the second mating end portion and movable toward the insertion end portion, and a third engaging ring portion configured to sleeve threadedly on the connecting portion of the catheter.

The image capturing unit is mounted on the insertion tube, and has an image capture device mounted on the insertion end portion, and a signal terminal signally connected to the image capture device and exposed from the second mating end portion. The image capture device is configured to capture image outside an end of the tubular body portion of the catheter. The signal terminal is configured to signally connect with the handgrip mechanism when the second mating end portion is connected to the handgrip mechanism.

Another further object of this disclosure is to provide a catheter that can alleviate at least one of the drawbacks of the prior art.

Accordingly, the catheter for mounting on the aforementioned endoscope device includes a connecting portion and a tubular body portion integrally connected to each other. The connecting portion is configured for the insertion end portion of the insertion tube to be inserted thereinto such that the connecting portion is sleeved on the insertion tube, is movable toward the connecting tube along the insertion tube so as to abut against and be limited by one of the first engaging ring portion and the second mating end portion, and is threadedly engageable with the second engaging ring portion. The tubular body portion is configured to receive the insertion tube when the insertion end portion is inserted into the connecting portion, and has an end opening for allowing the image capture device to capture image.

A still further object of this disclosure is to provide a catheter that can alleviate at least one of the drawbacks of the prior art.

Accordingly, the catheter for mounting on the aforementioned endoscope device includes a connecting portion and a tubular body portion integrally connected to each other. The connecting portion is configured for the insertion end portion of the insertion tube to be inserted thereinto such that the connecting portion is sleeved on the insertion tube, is movable toward the connecting tube along the insertion tube so as to abut against and be limited by one of the limiting ring portion of the third locking ring and the second mating end portion, and is threadedly engageable with the third engaging ring portion. The tubular body portion is configured to receive the insertion tube when the insertion end portion is inserted into the connecting portion, and has an end opening for allowing the image capture device to capture image.

### BRIEF DESCRIPTION OF THE DRAWINGS

Other features and advantages of the disclosure will become apparent in the following detailed description of the embodiments with reference to the accompanying drawings. It is noted that various features may not be drawn to scale.

FIG. 1 is a fragmentary side view of an endoscope device according to the first embodiment of the present disclosure.

FIG. 2 is a partly exploded and partly transparent view of FIG. 1.

FIG. 3 is a fragmentary transparent perspective view of a replaceable insertion tube mechanism of the first embodiment.

FIG. 4 is a fragmentary exploded perspective view of a handgrip mechanism and the replaceable insertion tube mechanism of the first embodiment.

FIG. 5 is a fragmentary sectional view of the handgrip mechanism and the replaceable insertion tube mechanism of the first embodiment in an assembled state.

FIG. 6 is a fragmentary exploded perspective view, illustrating the handgrip mechanism of the first embodiment capable of connecting with the replaceable insertion tube mechanisms of different outer diameters and lengths.

FIG. 7 is a view similar to FIG. 4, but illustrating another implementation of the first embodiment.

FIG. 8 is a fragmentary exploded perspective view of a handgrip mechanism and a replaceable insertion tube mechanism of an endoscope device according to the second embodiment of this disclosure.

FIG. 9 is a fragmentary sectional view of the handgrip mechanism and the replaceable insertion tube mechanism of the second embodiment in an assembled state.

FIG. 10 is a view similar to FIG. 9, but illustrating another implementation of the second embodiment.

FIG. 11 is a fragmentary exploded perspective view of a handgrip mechanism and a replaceable insertion tube mechanism of an endoscope device according to the third embodiment of the present disclosure.

FIG. 12 is a fragmentary sectional view of the handgrip mechanism and the replaceable insertion tube mechanism of the third embodiment in an assembled state and with a catheter sleeved on the replaceable insertion tube mechanism.

FIG. 13 is a view similar to FIG. 12, but illustrating another implementation of the third embodiment.

FIG. 14 is a fragmentary assembled sectional view of a handgrip mechanism and a replaceable insertion tube mechanism of the endoscope device according to the fourth embodiment of the present disclosure with a catheter sleeved on the replaceable insertion tube mechanism.

FIG. 15 is a fragmentary exploded sectional view of the handgrip mechanism and the replaceable insertion tube mechanism of the fourth embodiment and the catheter.

FIG. 16 is a view similar to FIG. 14, but illustrating another implementation of the fourth embodiment.

FIG. 17 is a fragmentary assembled sectional view of a handgrip mechanism and a replaceable insertion tube mechanism of the endoscope device according to the fifth embodiment of the present disclosure with a catheter sleeved on the replaceable insertion tube mechanism.

FIG. 18 is a view similar to FIG. 17, illustrating another implementation of the fifth embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

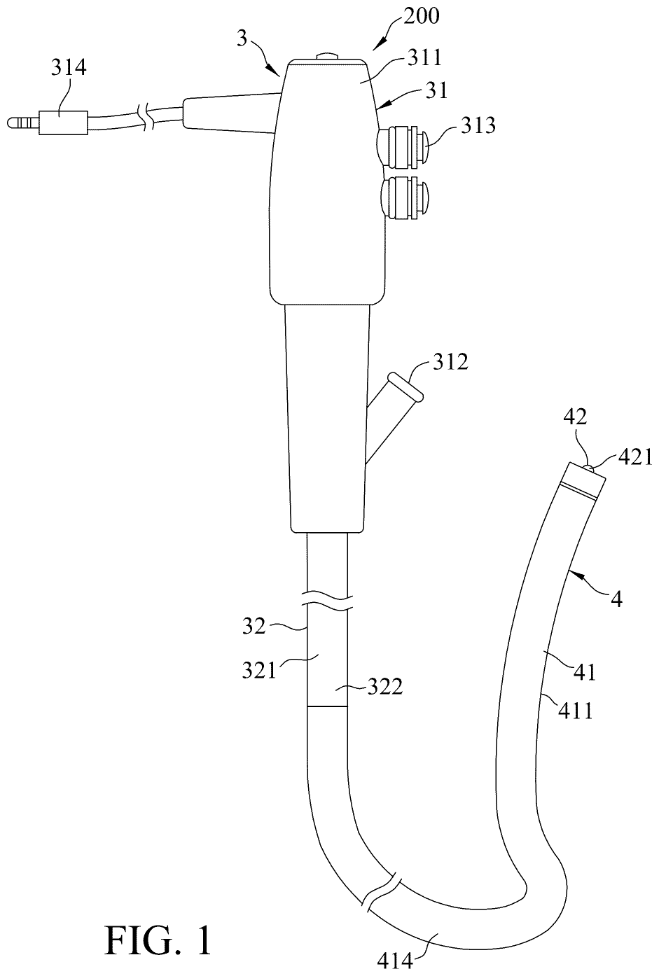

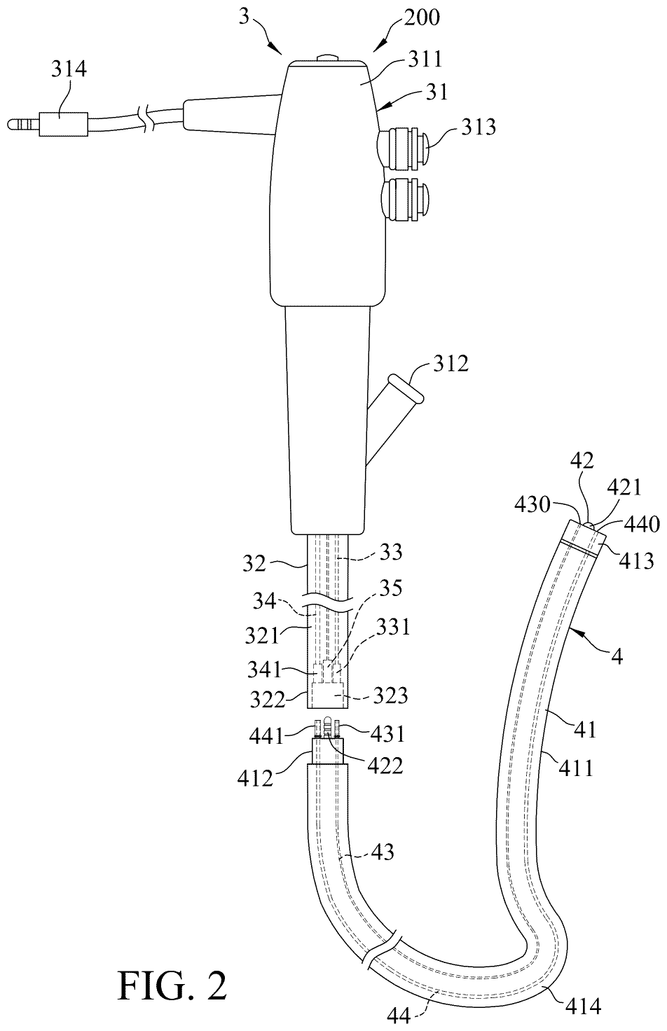

Referring to FIGS. 1 and 2, an endoscope device 200 according to the first embodiment of the present disclosure is configured to fluidly connect with a water supply device (not shown) and a suction device (not shown), is configured to signally connect with an image display apparatus (not shown), and is suitable for insertion into a catheter (not shown) to facilitate insertion operation thereof. The catheter may be, for example, but not limited to, a urinary catheter or a feeding tube. The endoscope device 200 comprises a handgrip mechanism 3 for connection with the water supply device, the suction device and the image display apparatus, and a replaceable insertion tube mechanism 4 detachably connected to the handgrip mechanism 3.

The handgrip mechanism 3 includes a handgrip unit 31, a connecting unit 32 extending outwardly from the handgrip unit 31, a first communicating tube 33 and a second communicating tube 34 insertedly disposed in the handgrip unit 31 and the connecting unit 32, and a signal connector 35 disposed in the connecting unit 32 and signally connected to the handgrip unit 31.

The handgrip unit 31 includes a handgrip body 311, and a water supply valve 312, a suction valve 313, and a communication module 314 all mounted on the handgrip body 311. The water supply valve 312 is fluidly connected to the second communicating tube 34, is configured to fluidly connect with the water supply device, and can be controlled to open so as to communicate the second communicating tube 34 with the water supply device. The suction valve 313 is fluidly connected to the first communicating tube 33, is configured to fluidly connect with the suction device, and can be controlled to open so as to communicate the first communicating tube 33 with the suction device. The communication module 314 is signally connected to the signal connector 35, and is configured to signally connect with the image display apparatus so as to signally connect the signal connector 35 to the image display apparatus.

In this embodiment, the communication module 314 is shown in the drawing as a transmission line for wired signal transmission. However, in other embodiments of this disclosure, the communication module 314 may be changed to a type that can be used for wireless communication with the image display apparatus, such as, but not limited to, a Bluetooth module.

The connecting unit 32 includes a connecting tube 321 extending outwardly from the handgrip body 311 and having a first mating end portion 322 distal to the handgrip body 311. An end surface of the first mating end portion 322 is recessed to form a mating groove 323 for insertion of the replaceable insertion tube mechanism 4 thereinto and be fixed thereat.

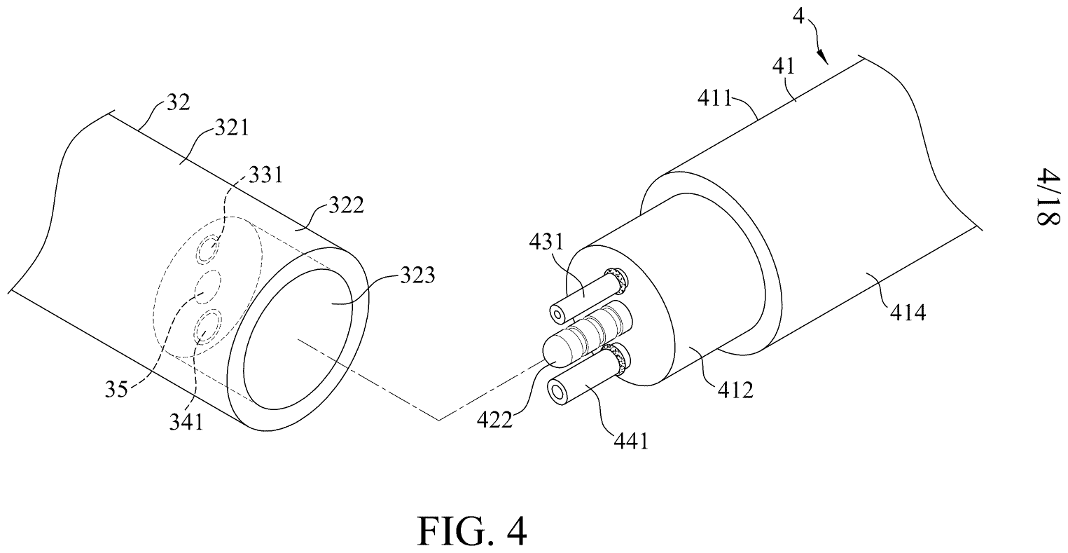

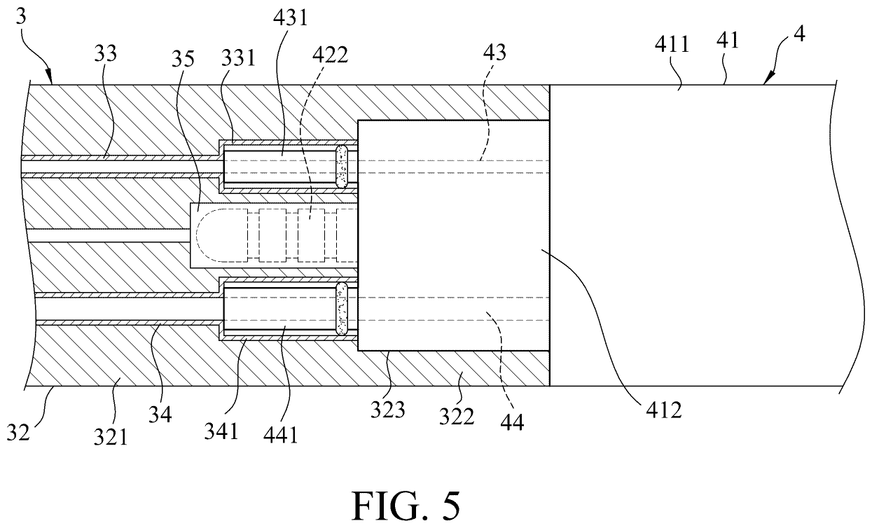

Referring to FIGS. 4 and 5, in combination with FIG. 2, the first communicating tube 33 is extended and inserted into the connecting tube 321, and has a first communication connector 331 coaxially communicating with and exposed in the mating groove 323. The second communicating tube 34 is extended and inserted into the connecting tube 321 spaced apart from the first communicating tube 33, and has a second communication connector 341 coaxially communicating with and exposed in the mating groove 323. The signal connector 35 is in the form of a socket disposed between the first and second communication connectors 331, 341 and communicating with and exposed in the mating groove 323.

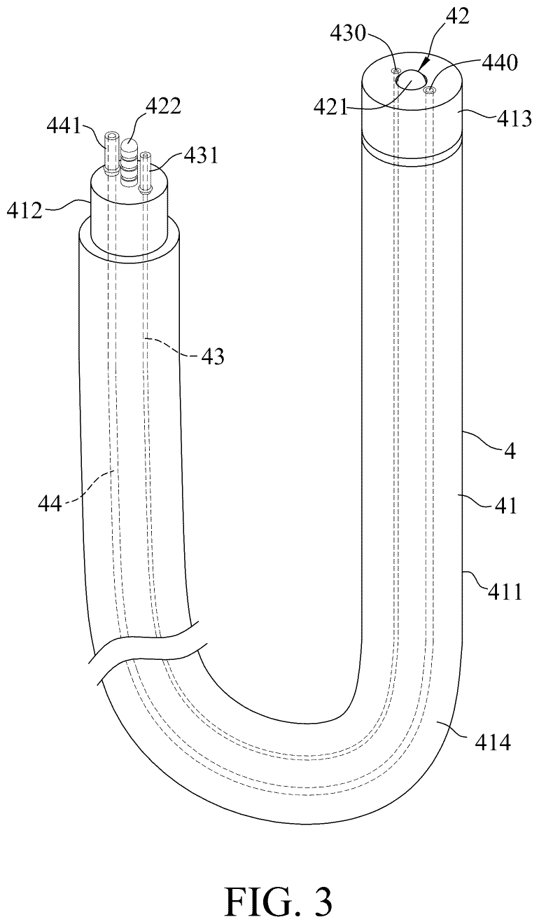

With reference to FIGS. 3 to 5, the replaceable insertion tube mechanism 4 is detachably connected to the first mating end portion 322, and includes an insertion tube unit 41, a first mating tube 43 and a second mating tube 44 inserted and disposed in the insertion tube unit 41, and an image capturing unit 42 disposed on the insertion tube unit 41.

In this embodiment, the insertion tube unit 41 includes an insertion tube 411 extending longitudinally and bendably deformable. The insertion tube 411 has a second mating end portion 412 and an insertion end portion 413 opposite to each other along a length thereof, and a main tube portion 414 connected between the second mating end portion 412 and the insertion end portion 413. The second mating end portion 412 has an outer diameter smaller than an outer diameter of the main tube portion 414.

The first mating tube 43 is extended and inserted into the insertion tube 411, and has one end provided with an opening 430 exposed on an end surface of the insertion end portion 413, and the other opposite end provided with a first mating connector 431 protruding out of an end surface of the second mating end portion 412. The second mating tube 44 is also extended and inserted into the insertion tube 411 spaced apart from the first mating tube 43, and has one end provided with an opening 440 exposed on the end surface of the insertion end portion 413 and spaced apart from the opening 430, and the other opposite end provided with a second mating connector 441 protruding from the end surface of the second mating end portion 412 and spaced apart from the first mating connector 431. The image capturing unit 42 has an image capture device 421 exposed on the end surface of the insertion end portion 413 between the openings 430 and 440 for capturing image, and a signal terminal 422 protruding from the end surface of the second mating end portion 412 between the first and second mating connectors 431 and 441 and signally connected to the image capture device 421. In this embodiment, the image capture device 421 is, for example, but not limited to, a charge-coupled device (CCD).

Referring again to FIGS. 2, 4 and 5, to assemble the handgrip mechanism 3 and the replaceable insertion tube mechanism 4, the second mating end portion 412 of the insertion tube 411 is detachably and coaxially inserted into the mating groove 323 of the first mating end portion 322 of the connecting tube 321, and brings the first mating connector 431 to tightly insert into the first communication connector 331, the second mating connector 441 to tightly insert into the second communication connector 341, and the signal terminal 422 to tightly insert into the signal connector 35, thereby completing the assembly.

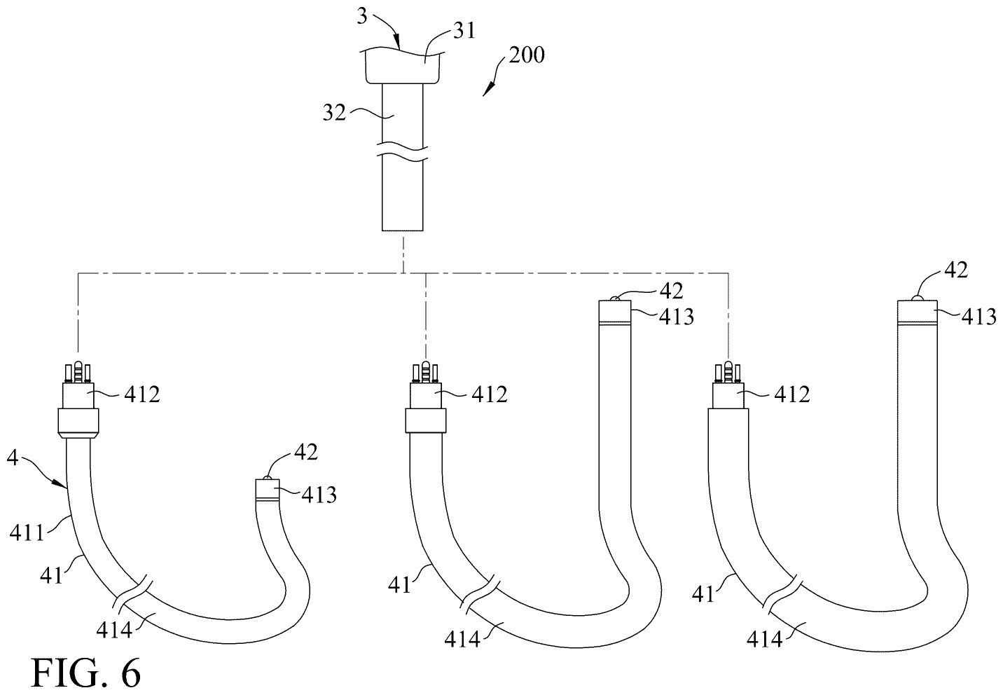

Referring to FIG. 6, in combination with FIG. 1, in practice, the main tube portion 414 and the insertion end portion 413 of the insertion tube 411 may be manufactured to have different outer diameters or lengths according to various diameters of catheters. When it is desired to replace the insertion tube 411 with a different diameter or length, the replaceable insertion tube mechanism 4 is simply detached from the connecting tube 321, after which another replaceable insertion tube mechanism 4 having the desired outer diameter or length of the insertion tube 411 is inserted into the connecting tube 321.

When the endoscope device 200 of this disclosure is used for performing insertion guidance of a catheter, such as a nasogastric tube, a nasojejunal tube, a nasal feeding tube, a naso-enteric tube, etc., and when the catheter is not provided with a guide wire (not shown), according to an inner diameter of the catheter to be used, the insertion tube unit 41 having the insertion tube 411 of a suitable size is mounted to the handgrip mechanism 3, and the insertion tube 411 is inserted into the catheter. On one hand, the insertion tube 411 can be used to increase the supporting force of the catheter, making it easy to push forward the catheter. On the other hand, the image capture device 421 disposed on the insertion tube 411 can be adjacent to a front end opening of the catheter, and can be used to capture image in the forward advance direction of the catheter.

The image captured by the image capture device 421 can then be transmitted to an image output apparatus signally connected to the communication module 314 through the signal terminal 422 and the signal connector 35. Consequently, during the insertion process of the catheter, the medical personnel can immediately see an image of the body cavity in front of the catheter, thereby facilitating adjustment of the advance direction or observation of tissue of the body cavity.

When it is required to suction the body cavity, for example, suction of water or gas, the suction valve 313 can be controlled to open, and a negative pressure suction force generated by the suction device through fluid connection with the first communicating tube 33 and the first mating tube 43 can be used to suction the water or gas in the cavity in front of the insertion end portion 413. When it is required to flush the body cavity in which the insertion operation is performed, the water supply valve 312 can be controlled to open, and water liquid from the water supply device can be guided and sprayed toward the front of the insertion end portion 413 through the second communicating tube 34 and the second mating tube 44.

By inserting the insertion tube 411 of the endoscope device 200 into the catheter to perform the insertion operation of the latter, the disadvantages of arranging the insertion tube of the existing endoscope device side by side with the catheter can be resolved.

Similarly, when an insertion operation of a urinary catheter is to be performed, based on an inner diameter of the urinary catheter used, the insertion tube unit 41 having the insertion tube 411 of suitable outer diameter and length is mounted on the handgrip mechanism 3, and the insertion tube 411 is inserted into the urinary catheter such that the image capture device 421 is adjacent to a front opening of the urinary catheter and can be used to capture image in the forward advancing direction of the urinary catheter. Through this configuration, during the insertion process of the urinary catheter, the swelling condition of the prostate can be immediately seen and evaluated through image monitoring, which can help the medical personnel to evaluate and directly replace the urinary catheter with an appropriate size. In addition, by virtue of the design of the insertion tube 411 of the endoscope device 200 being insertable into the urinary catheter, the supporting strength of the urinary catheter during insertion can be improved. Therefore, the urinary catheter can be made of softer tubing to help improve the discomfort caused by high hardness of the existing urinary catheter.

Referring once again to FIGS. 3 to 5, in the first embodiment, the first mating end portion 322 is recessed to form the mating groove 323, so that the first and second mating end portions 322, 412 can be inserted into each other for assembly. However, in other embodiments of this disclosure, the second mating end portion 412 may be recessed to form the mating groove, so that the first mating end portion 322 may be inserted into the mating groove of the second mating end portion 412 for assembly.

In addition, in the first embodiment, the first and second communication connectors 331, 341 and the signal connector 35 are designed to be embedded in the first mating end portion 322 and are exposed in the mating groove 323, and the first and second mating connectors 431, 441 and the signal terminal 422 are designed to protrude out of the end surface of the second mating end portion 412, so that, when the first and second mating end portions 322, 412 are mated to each other, the first and second mating connectors 431, 441 and the signal terminal 422 can be respectively inserted into the first and second communication connectors 331, 341 and the signal connector 35. However, in other embodiments of this disclosure, the first and second communication connectors 331, 341 and the signal connector 35 may be configured to protrude into the mating groove 323 or protrude out of the end surface of the first mating end portion 322, while the first and second mating connectors 431, 441 and the signal terminal 422 may be configured to be embedded in the second mating end portion 412 and exposed on the end surface of the second mating end portion 412 or in the mating groove of the second mating end portion. In this case, when the first and second mating end portions 322, 412 are mated to each other, the first and second communication connectors 331, 341 and the signal connector 35 may be respectively inserted into the first and second mating connectors 431, 441 and the signal terminal 422. Likewise, water can be guided and suctioned, and signal transmission can be performed.

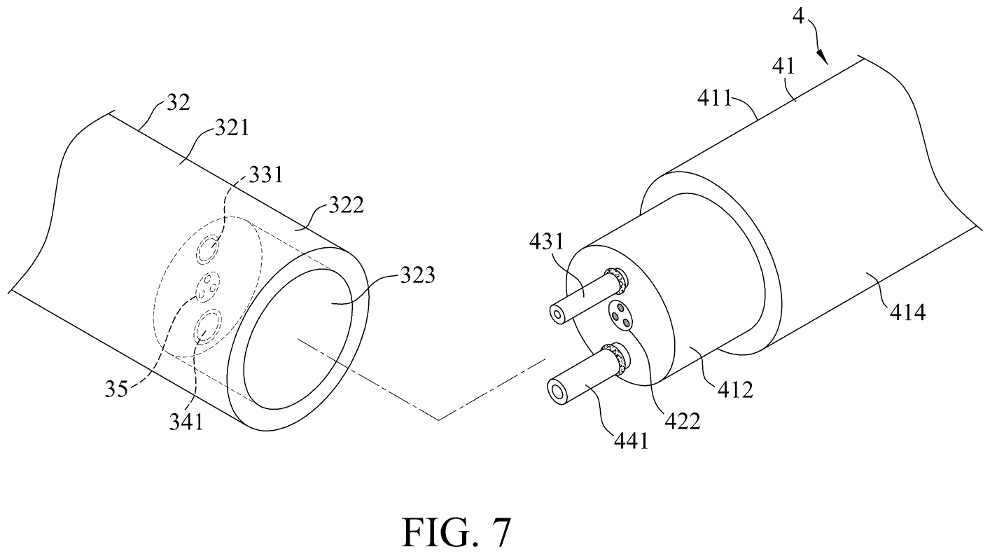

Referring to FIG. 7, furthermore, in another implementation of this embodiment, the signal connector 35 and the signal terminal 422 may not be inserted into each other, but may be designed as planar shapes, so that, when the first and second mating end portions 322, 412 are mated to each other, they are in electrical contact with each other for transmission of electrical power and signal.

Since there are many methods of assembling the first and second mating end portions 322, 412, there are also many methods of connecting the first and second communication connectors 331, 341 with the first and second mating connectors 431, 441, respectively, and there are also many methods of signally connecting the signal connector 35 with the signal terminal 422, the connections thereof are not limited to the aforesaid disclosed embodiment.

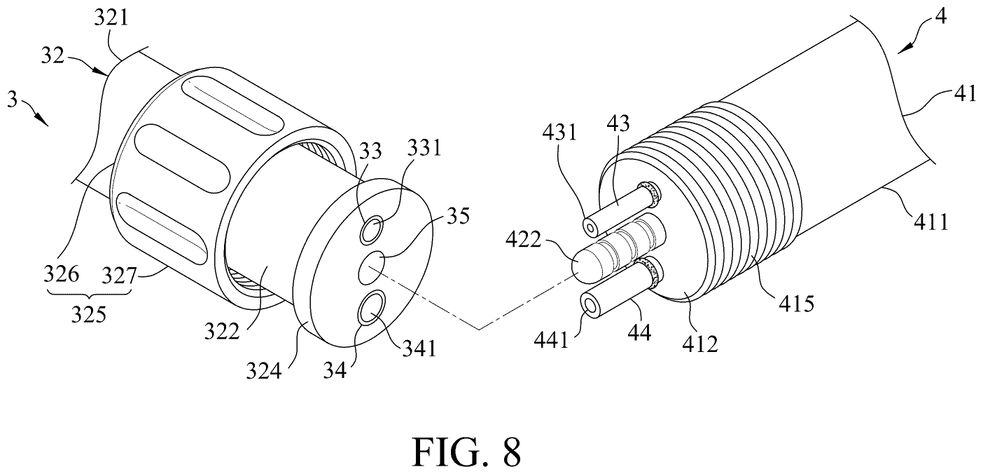

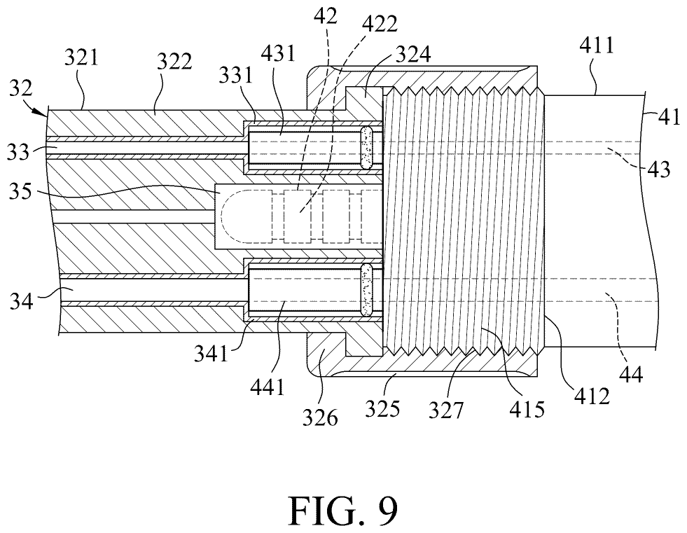

Referring to FIGS. 8 and 9, the second embodiment of the endoscope device 200 of this disclosure is substantially similar to the first embodiment, but differs from the first embodiment in the assembly structure of the connecting unit 32 and the insertion tube unit 41. For convenience of description, only the differences between the first and second embodiments will be described hereinafter.

In the second embodiment, the connecting unit 32 further includes a first locking ring 325 sleeved over the first mating end portion 322 and movable along a length of the connecting tube 321. The first mating end portion 322 is not formed with the mating groove 323 (see FIG. 2). The connecting tube 321 further has an abutment ring portion 324 projecting radially and outwardly from an outer periphery of the first mating end portion 322. The first locking ring 325 has a limiting ring portion 326 movable toward the first mating end portion 322 and be blocked and limited by the abutment ring portion 324, and a first engaging ring portion 327 extending coaxially and outwardly from the limiting ring portion 326 across the abutment ring portion 324 and protruding out of the first mating end portion 322. The first engaging ring portion 327 has an inner peripheral surface provided with threads.

The first and second communication connectors 331, 341 and the signal connector 35 are exposed on an end surface of the first mating end portion 322. An outer peripheral surface of the second mating end portion 412 of the insertion tube 411 is provided with threads 415.

To assemble the handgrip mechanism 3 and the replaceable insertion tube mechanism 4, the first and second mating connectors 431, 441 and the signal terminal 422 protruding out of the second mating end portion 412 are respectively inserted into the first and second communication connectors 331, 341 and the signal connector 35, until the first and second mating end portions 322, 412 are in end-to-end contact with each other. Then, the first engaging ring portion 327 of the first locking ring 325 is threadedly engaged with the threads 415 of the second mating end portion 412, and the limiting ring portion 326 is driven to move along with the first engaging ring portion 327 toward the first mating end portion 322 until the limiting ring portion 326 abuts against and is limited by the abutment ring portion 324, thereby completing the assembly of the handgrip mechanism 3 and the replaceable insertion tube mechanism 4.

When it is required to replace other replaceable insert tube mechanisms 4, it is only necessary to threadedly disengage the first locking ring 325 from the second mating end portion 412 so as to detach the replaceable insertion tube mechanism 4 from the handgrip mechanism 3.

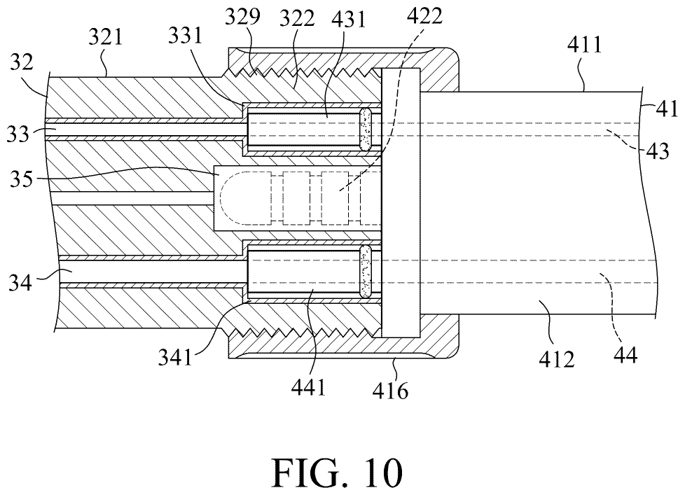

In the second embodiment, the first locking ring 325 is sleeved over the connecting tube 321, while the outer peripheral surface of the second mating end portion 412 is provided with the threads 415 for threaded engagement with the first engaging ring portion 327 of the first locking ring 325. However, in another implementation of this embodiment, the connection structure of the first locking ring 325 and the threads 415 of the second mating end portion 412 may be interchanged. That is, as shown in FIG. 10, the connecting unit 32 may not be provided with the first locking ring 325, and an outer peripheral surface of the first mating end portion 322 of the connecting tube 321 may be provided with threads 329. Moreover, the insertion tube unit 41 may be designed to further include a second locking ring 416 sleeved over the second mating end portion 412 of the insertion tube 411 and having an inner peripheral surface provided with threads, so that the threads of the second locking ring 416 may be threadedly engaged with the threads 329 of the first mating end portion 322. Through this configuration, the connecting tube 321 and the insertion tube 411 may similarly be in end-to-end contact with each other.

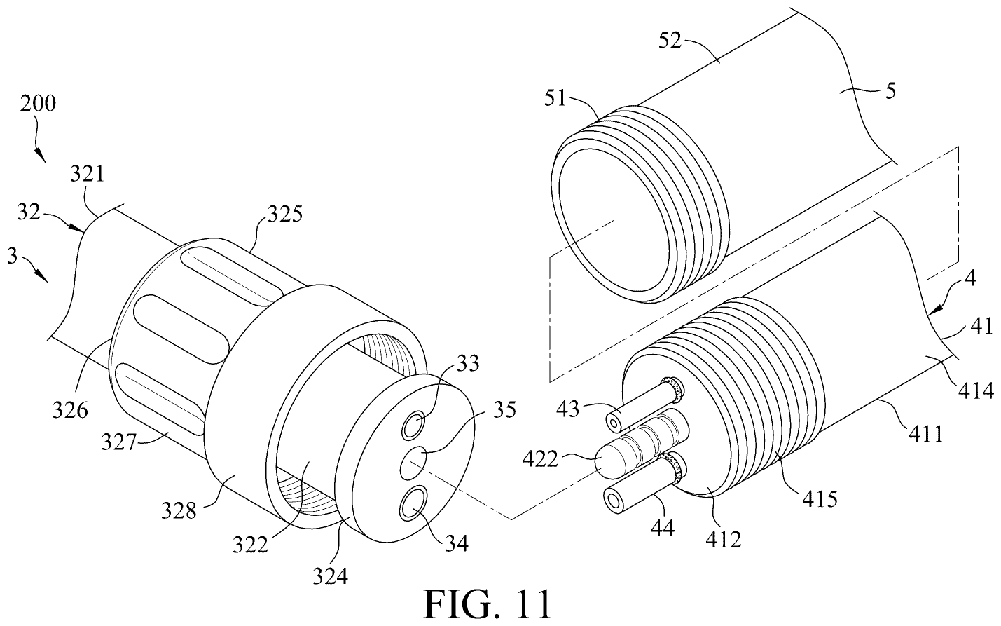

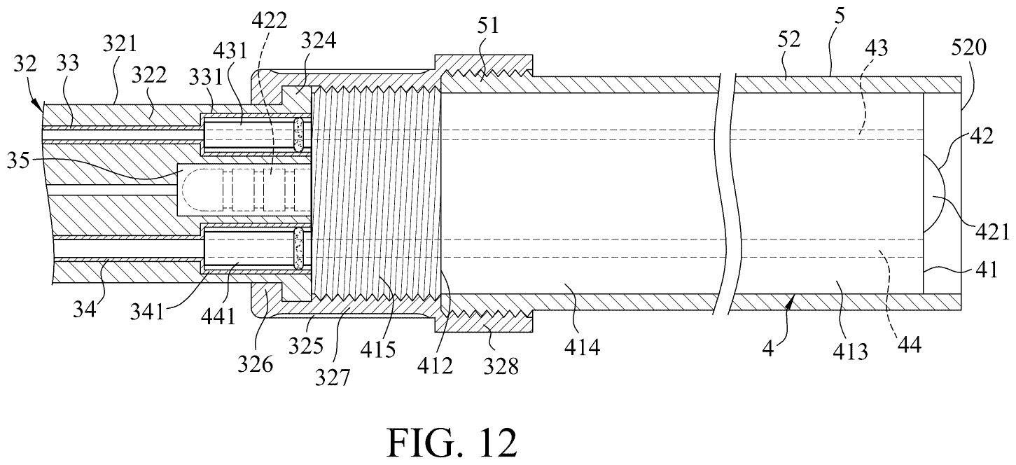

Referring to FIGS. 11 and 12, the third embodiment of the endoscope device 200 of this disclosure is substantially similar to the second embodiment, but differs from the second embodiment in the structural design of the first locking ring 325. Furthermore, the endoscope device 200 of this embodiment is also suitable for connection with a catheter 5 which includes a connecting portion 51 and a tubular body portion 52 integrally connected to each other. An outer peripheral surface of the connecting portion 51 is provided with threads. The tubular body portion 52 has an end opening 520.

The first locking ring 325 of this embodiment further has a second engaging ring portion 328 extending coaxially and outwardly from the first engaging ring portion 327 and having an inner diameter greater than an inner diameter of the first engaging ring portion 327. The second engaging ring portion 328 is configured to spacedly surround the main tube portion 414.

When the endoscope device 200 is used to mount the catheter 5, the insertion tube 411 is inserted through the insertion end portion 413 thereof from the connecting portion 51 to the tubular body portion 52 of the catheter 5, such that the main tube portion 414 is extended and inserted into the tubular body portion 52 of the catheter 5, until the connecting portion 51 is adjacent to the second mating end portion 412. At this time, the insertion end portion 413 of the insertion tube 411 is adjacent to the end opening 520 of the tubular body portion 52, so that the image capture device 421 can capture image outside the end opening 520, the second mating tube 44 can spray water liquid outwardly through the end opening 520, and the first mating tube 43 can suction water liquid and gas through the end opening 520.

Next, the first locking ring 325 is moved toward the insertion tube 411 so as to threadedly connect the second engaging ring portion 328 with the connecting portion 51 and the first engaging ring portion 327 with the second mating end portion 412 until the connecting portion 51 abuts against an end of the first engaging ring portion 327 and is limited thereby, and the limiting ring portion 326 abuts against the abutment ring portion 324 and is limited thereby.

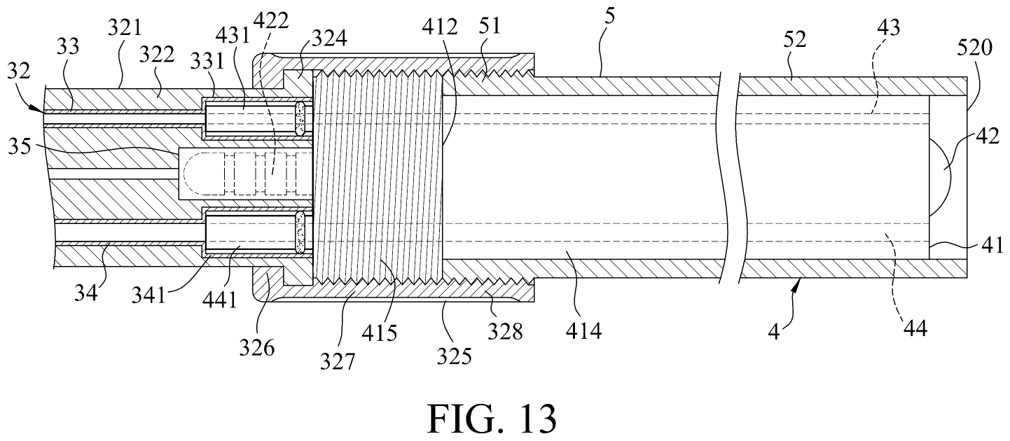

In the third embodiment, the second mating end portion 412 and the main tube portion 414 of the insertion tube 411 have equal outer diameters (see FIG. 12), so that, when the catheter 5 is sleeved on the main tube portion 414 of the insertion tube 411, an outer diameter of the connecting portion 51 is greater than the outer diameter of the second mating end portion 412, and the inner diameter of the second engaging ring portion 328 is greater than the inner diameter of the first engaging ring portion 327. However, in another implementation of this embodiment, the outer diameter of the main tube portion 414 of the insertion tube 411 may be designed to be smaller than the outer diameter of the second mating end portion 412, as shown in FIG. 13, so that, when the catheter 5 is sleeved on the main tube portion 414, the connecting portion 51 and the second mating end portion 412 have equal outer diameters. Furthermore, the insertion tube 411 may use the second mating end portion 412 for abutment with and for limiting the connecting portion 51 when the latter moves toward the connecting tube 321, so that the first and second engaging ring portions 327, 328 of the first locking ring 325 may be designed to have equal inner diameters.

Through the structural design of the third embodiment that can be used to connect with the catheter 5, the endoscope device 200 of this disclosure can be directly and fixedly connected to the catheter 5 so as to facilitate insertion and guidance operation of the catheter 5, thereby improving the insertion success rate of the catheter 5 and the medical quality.

It should be noted that, in the aforementioned embodiments, suction and flushing are respectively performed through the interconnection of the first communicating tube 33 and the first mating tube 43 and the interconnection of the second communicating tube 34 and the second mating tube 44. However, in other embodiments of this disclosure, the second communicating tube 34 and the second mating tube 44 may not be provided, and the first communicating tube 33 may fluidly connect with the water supply valve 312 and the suction valve 313. In this case, after the first communicating tube 33 is connected to the first mating tube 43, the first communicating tube 33 can perform flushing and suction operations through a single pipeline by respectively operating the water supply valve 312 and the suction valve 313.

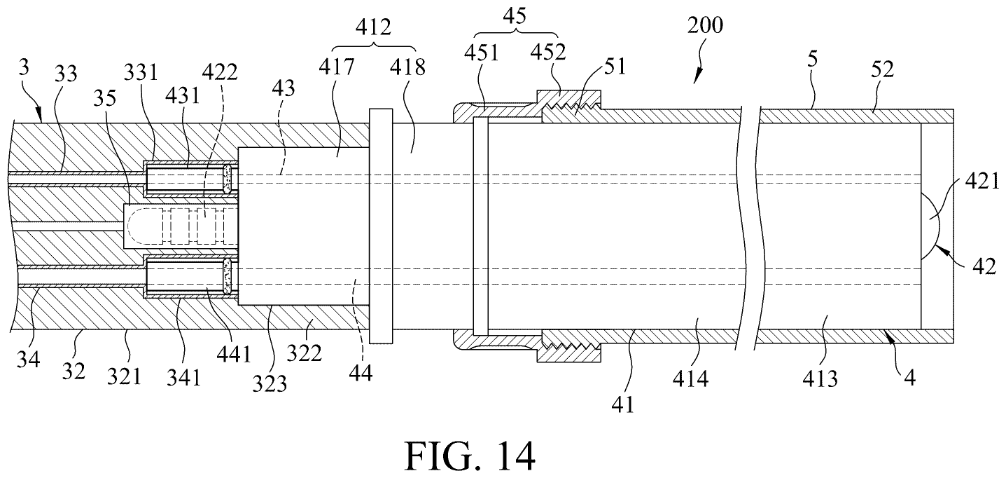

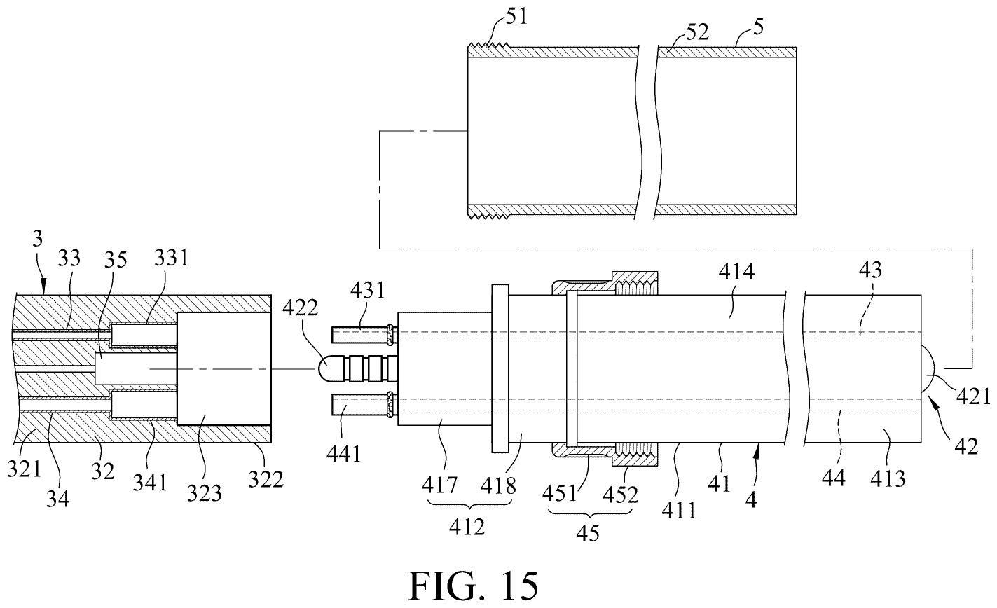

Referring to FIGS. 14 and 15, the fourth embodiment of the endoscope device 200 of this disclosure is substantially similar to the third embodiment, but differs from the third embodiment in that the connecting unit 32 is not provided with the first locking ring 325 (see FIG. 11) for connection with the catheter 5, and the replaceable insertion tube mechanism 4 further includes a third locking ring 45 for connection with the catheter 5.

In the fourth embodiment, the second mating end portion 412 has a mating section 417, and a connecting section 418 connected between the mating section 417 and the main tube portion 414. The first and second mating connectors 431, 441 and the signal terminal 422 all protrude from and are exposed on an end surface of the mating section 417. The replaceable insertion tube mechanism 4 is assembled to the handgrip mechanism 3 through detachable connection of the mating section 417 of the second mating end portion 412 and the mating groove 323 of the first mating end portion 322, so that the first and second mating tubes 43, 44 can respectively communicate with the first and second communicating tubes 33, 34, and so that the signal terminal 422 can be signally connected to the signal connector 35. Since this assembling method is similar to that described in the third embodiment, a detailed description thereof is dispensed herewith.

The third locking ring 45 is sleeved on the connecting section 418, is movable along a length of the insertion tube 411, and has a limiting ring portion 451 rotatably sleeved on the second mating end portion 412 and movable toward the insertion end portion 413, and a third engaging ring portion 452 radially expanded relative to the limiting ring portion 451 and extending toward the insertion end portion 413.

When the replaceable insertion tube mechanism 4 is used to assemble with the catheter 5, the insertion tube 411 is inserted through the insertion end portion 413 thereof from the connecting portion 51 to the tubular body portion 52 of the catheter 5, until the connecting portion 51 is relatively close to the second mating end portion 412. Thereafter, the third locking ring 45 is threadedly rotated toward the catheter 5, and the third engaging ring portion 452 thereof is threadedly connected to the threads of the connecting portion 51 until the connecting portion 51 abuts against the limiting ring portion 451 and is positioned thereat. At this time, the catheter 5 is fixedly sleeved on the insertion tube 411.

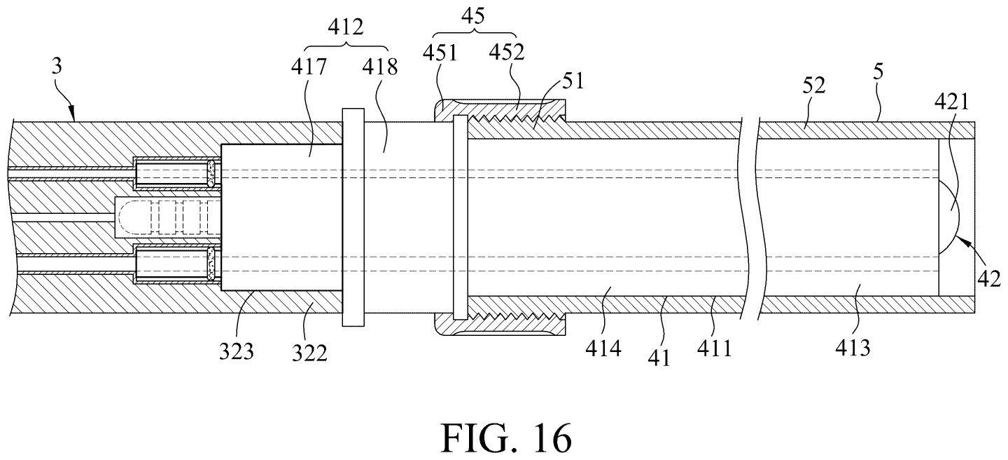

Referring to FIG. 16, in another implementation of this embodiment, when an outer diameter of the main tube portion 414 of the insertion tube 411 is smaller than an outer diameter of the second mating end portion 412, the connecting portion 51 of the catheter 5 may be designed to have the same outer diameter as the second mating end portion 412. As such, when the catheter 5 is sleeved on the insertion tube 411, the connecting portion 51 can abut against and be limited by the connecting section 418 of the second mating end portion 412 when moved toward the connecting unit 32. In this case, the limiting ring portion 451 and the third engaging ring portion 452 of the third locking ring 45 can be designed to have equal outer diameters and threadedly connected to the connecting portion 51.

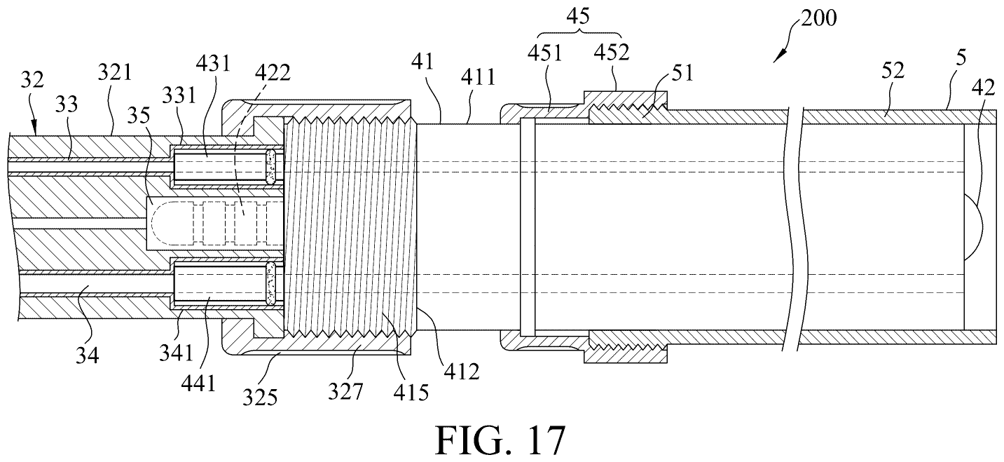

Referring to FIG. 17, the fifth embodiment of the endoscope device 200 of this disclosure is substantially similar to the fourth embodiment, but differs from the fourth embodiment in the assembly structure of the connecting unit 32 and the insertion tube unit 41.

In the fifth embodiment, the assembly structure of the connecting unit 32 and the insertion tube unit 41 is similar to that described in the second embodiment. That is, in the fifth embodiment, the connection structure of the third locking ring 45 and the catheter 5 described in the fourth embodiment is combined with the assembly structure of the connecting unit 32 and the insertion tube unit 41 described in the second embodiment and as shown in FIG. 9, so that assembly between the connecting unit 32 of the handgrip mechanism 3 and the insertion tube unit 41 of the replaceable insertion tube mechanism 4 can be achieved through the first locking ring 325, and the insertion tube unit 41 and the catheter 5 can be assembled through the third locking ring 45.

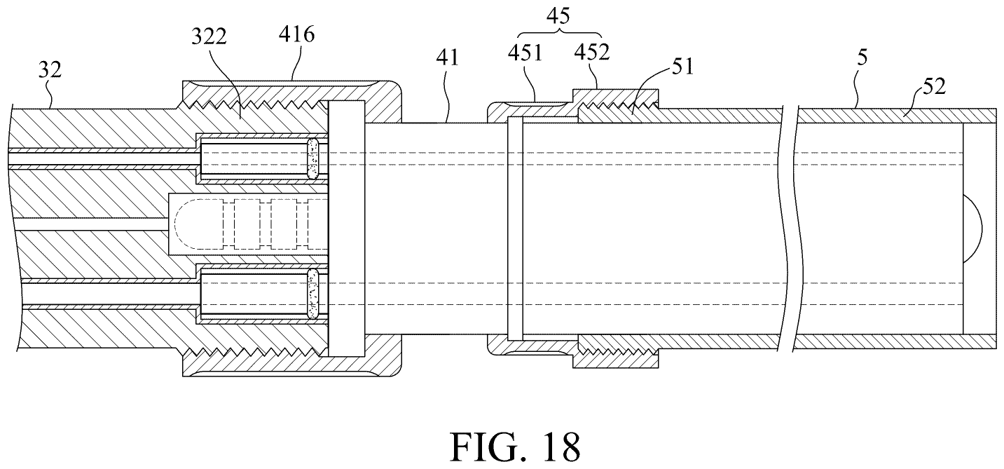

Referring to FIG. 18, another implementation of the fifth embodiment is to combine the connection structure of the third locking ring 45 and the catheter 5 described in the fourth embodiment with the assembly structure of the connecting unit 32 and the insertion tube unit 41 described in the another implementation of the second embodiment and as shown in FIG. 10, so that assembly between the connecting unit 32 of the handgrip mechanism 3 and the insertion tube unit 41 of the replaceable insertion tube mechanism 4 can be achieved through the second locking ring 416. Similarly, the insertion tube unit 41 and the catheter 5 can be assembled through the third locking ring 45.

In summary, through the detachable assembly structure of the handgrip mechanism 3 and the replaceable insertion tube mechanism 4, the medical personnel can be facilitated to select and connect the replaceable insertion tube mechanism 4 with the required outer diameter or length to the handgrip mechanism 3 according to various usage requirements. That is, one handgrip mechanism 3 can be used with a plurality of replaceable insert tube mechanisms 4 having insertion tubes 411 of different outer diameters or lengths, and, once the replaceable insertion tube mechanism 4 currently used by the endoscope device 200 is damaged, only the replaceable insertion tube mechanism 4 needs to be replaced, and there is no need to replace the entire endoscope device 200. Hence, cost of purchasing and installing the endoscope device 200 by the medical institution can be reduced.

Furthermore, through the structural design of the first locking ring 325 or the third locking ring 45, the catheter 5 sleeved on the insertion tube 411 can be fixed, so that the endoscope device 200 can be disposed coaxially with the catheter 5 to assist in the insertion and guidance of the catheter 5. This can improve the current disadvantage of arranging the insertion tube of the endoscope device outside and side by side with the catheter for carrying out the insertion and guidance operation.

Moreover, through the structural design of the connecting portion 51 of the catheter 5 for threaded engagement with the first locking ring 325 or the third locking ring 45, the catheter 5 can be stably mounted to the endoscope device 200, and can synchronously move with the replaceable insertion tube mechanism 4, so as to enhance the success rate of insertion.

Therefore, the endoscope device 200 of this disclosure having the replaceable insertion tube mechanism 4 and the catheter 5 used for the endoscope device 200 are indeed innovative and convenient to use, and can achieve the object of this disclosure.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. An endoscope device suitable for connection with a catheter, the catheter including a connecting portion and a tubular body portion, said endoscope device comprising:
a handgrip mechanism including a handgrip unit, a connecting unit connected to said handgrip unit and having a first mating end portion distal to said handgrip unit, and a signal connector signally connected to said handgrip unit and exposed from said first mating end portion, said connecting unit including a connecting tube having said first mating end portion, and a first locking ring sleeved over said first mating end portion and movable along a length of said connecting tube; and
a replaceable insertion tube mechanism including an insertion tube unit having a second mating end portion and an insertion end portion opposite to each other along a length of said insertion tube unit, and an image capturing unit mounted on said insertion tube unit, said insertion tube unit including an insertion tube having said second mating end portion and said insertion end portion and being configured to be insertable through said insertion end portion from the connecting portion to the tubular body portion of the catheter, said image capturing unit having an image capture device mounted on said insertion end portion, and a signal terminal signally connected to said image capture device and exposed from said second mating end portion, said second mating end portion being detachably and coaxially connected to said first mating end portion, and bringing said signal terminal to detachably and signally connect with said signal connector, said image capture device being configured to capture image outside an end of the tubular body portion of the catheter;
said first locking ring being detachably and threadedly connected to said second mating end portion so as to secure said first mating end portion and said second mating end portion in an end-to-end manner, said first locking ring having a first engaging ring portion sleeved threadedly on said second mating end portion, and a second engaging ring portion configured to sleeve threadedly on the connecting portion of the catheter.

2. The endoscope device as claimed in claim 1, wherein said signal connector is exposed on an end surface of said first mating end portion, and said signal terminal is exposed on an end surface of said second mating end portion and is electrically connected to said signal connector.

3. The endoscope device as claimed in claim 2, wherein said handgrip mechanism further includes a first communicating tube disposed in said handgrip unit and extended and inserted into said connecting tube, said first communicating tube having a first communication connector exposed on said end surface of said first mating end portion, said replaceable insertion tube mechanism further including a first mating tube extended and inserted into said insertion tube, said first mating tube having a first mating connector exposed on said end surface of said second mating end portion and detachably connected to said first communication connector.

4. The endoscope device as claimed in claim 3, wherein said handgrip mechanism further includes a second communicating tube disposed in said handgrip unit and extended and inserted into said connecting tube, said second communicating tube having a second communication connector exposed on said end surface of said first mating end portion, said replaceable insertion tube mechanism further including a second mating tube extended and inserted into said insertion tube, said second mating tube having a second mating connector exposed on said end surface of said second mating end portion and detachably connected to said second communication connector.

5. The endoscope device as claimed in claim 4, wherein said handgrip unit includes a handgrip body, a water supply valve mounted on said handgrip body and fluidly connected to said second communicating tube, a suction valve mounted on said handgrip body and fluidly connected to said first communicating tube, and a communication module mounted on said handgrip body and signally connected to said signal connector.

6. The endoscope device as claimed in claim 1, wherein said insertion tube further has a main tube portion connected between said second mating end portion and said insertion end portion, said second mating end portion having an outer diameter greater than an outer diameter of said main tube portion, said insertion tube being configured to be insertable through said insertion end portion from the connecting portion to the tubular body portion of the catheter such that said main tube portion is extended and inserted into the tubular body portion of the catheter, said second mating end portion being configured for abutment with and for limiting the connecting portion of the catheter when the connecting portion moves toward said connecting tube.

7. The endoscope device as claimed in claim 1, wherein said connecting tube further has an abutment ring portion projecting radially and outwardly from an outer periphery of said first mating end portion, said first locking ring further having a limiting ring portion connected to said first engaging ring portion, said limiting ring portion being driven to move along with said first engaging ring portion toward said first mating end portion until said limiting ring portion is blocked and limited by said abutment ring portion.

8. The endoscope device as claimed in claim 7, wherein an inner diameter of said second engaging ring portion is greater than an inner diameter of said first engaging ring portion.

9. The endoscope device 200 as claimed in claim 7, wherein said second engaging ring portion and said first engaging ring portion have equal inner diameters.

10. An endoscope device suitable for connection with a catheter, the catheter including a connecting portion and a tubular body portion, said endoscope device comprising:
a handgrip mechanism including a handgrip unit, a connecting unit connected to said handgrip unit and having a first mating end portion distal to said handgrip unit, and a signal connector signally connected to said handgrip unit and exposed from said first mating end portion, said connecting unit including a connecting tube having said first mating end portion; and
a replaceable insertion tube mechanism including an insertion tube unit, and an image capturing unit mounted on said insertion tube unit, said insertion tube unit including an insertion tube having a second mating end portion and an insertion end portion opposite to each other along a length of said insertion tube (411), and a third locking ring sleeved on said second mating end portion and movable along the length of said insertion tube, said image capturing unit having an image capture device mounted on said insertion end portion, and a signal terminal signally connected to said image capture device and exposed from said second mating end portion, said second mating end portion being detachably and coaxially connected to said first mating end portion, and bringing said signal terminal to detachably and signally connect with said signal connector, said insertion tube being configured to be insertable through said insertion end portion from the connecting portion to the tubular body portion of the catheter, said image capture device being configured to capture image outside an end of the tubular body portion of the catheter, said third locking ring having a limiting ring portion sleeved on said second mating end portion and movable toward said insertion end portion, and a third engaging ring portion configured to sleeve threadedly on the connecting portion of the catheter.

11. The endoscope device as claimed in claim 10, wherein said third locking ring is rotatably sleeved on said second mating end portion.

12. The endoscope device as claimed in claim 10, wherein said limiting ring portion of said third locking ring (45) is configured for abutment with and for limiting the connecting portion of the catheter when the connecting portion moves toward said connecting unit.

13. The endoscope device as claimed in claim 10, wherein said insertion tube further has a main tube portion connected between said second mating end portion and said insertion end portion, said second mating end portion having an outer diameter greater than an outer diameter of said main tube portion, and being configured for abutment with and for limiting the connecting portion of the catheter when the connecting portion moves toward said connecting unit.

14. An endoscope device suitable for connection with a catheter, the catheter including a connecting portion and a tubular body portion, said endoscope device comprising:
a handgrip mechanism including a handgrip unit, a connecting unit connected to said handgrip unit and having a first mating end portion distal to said handgrip unit, and a signal connector signally connected to said handgrip unit and exposed from said first mating end portion, said connecting unit including a connecting tube having said first mating end portion, and a first locking ring sleeved over said first mating end portion and movable along a length of said connecting tube; and
a replaceable insertion tube mechanism including an insertion tube unit having a second mating end portion and an insertion end portion opposite to each other along a length of said insertion tube unit, and an image capturing unit mounted on said insertion tube unit, said image capturing unit having an image capture device mounted on said insertion end portion, and a signal terminal signally connected to said image capture device and exposed from said second mating end portion, said image capture device being configured to capture image outside an end of the tubular body portion of the catheter, said insertion tube unit including an insertion tube having said second mating end portion and said insertion end portion, and a third locking ring sleeved on said second mating end portion and movable along a length of said insertion tube;
said second mating end portion being detachably and coaxially connected to said first mating end portion, and bringing said signal terminal to detachably and signally connect with said signal connector, said first locking ring being detachably and threadedly connected to said second mating end portion so as to secure said first mating end portion and said second mating end portion in an end-to-end manner, said insertion tube being configured to be insertable through said insertion end portion from the connecting portion to the tubular body portion of the catheter, said third locking ring having a limiting ring portion sleeved on said second mating end portion and movable toward said insertion end portion, and a third engaging ring portion configured to sleeve threadedly on the connecting portion of the catheter.

15. The endoscope device as claimed in claim 14, wherein said third locking ring is rotatably sleeved on said second mating end portion.

16. The endoscope device as claimed in claim 14, wherein said limiting ring portion of said third locking ring is configured for abutment with and for limiting the connecting portion of the catheter when the connecting portion moves toward said connecting tube.

17. The endoscope device as claimed in claim 14, wherein said insertion tube further has a main tube portion connected between said second mating end portion and said insertion end portion, said second mating end portion having an outer diameter greater than an outer diameter of said main tube portion, and being configured for abutment with and for limiting the connecting portion of the catheter when the connecting portion moves toward said connecting tube.

18. An endoscope device suitable for connection with a catheter, the catheter including a connecting portion and a tubular body portion, said endoscope device comprising:
a handgrip mechanism including a handgrip unit, a connecting unit connected to said handgrip unit and having a first mating end portion distal to said handgrip unit, and a signal connector signally connected to said handgrip unit and exposed from said first mating end portion, said connecting unit including a connecting tube having said first mating end portion; and
a replaceable insertion tube mechanism including an insertion tube unit having a second mating end portion and an insertion end portion opposite to each other along a length of said insertion tube unit, and an image capturing unit mounted on said insertion tube unit, said image capturing unit having an image capture device mounted on said insertion end portion, and a signal terminal signally connected to said image capture device and exposed from said second mating end portion, said image capture device being configured to capture image outside an end of the tubular body portion of the catheter, said insertion tube unit including an insertion tube having said second mating end portion and said insertion end portion, a second locking ring sleeved over said second mating end portion and movable along a length of said insertion tube, and a third locking ring sleeved on said second mating end portion and movable along the length of said insertion tube, said second mating end portion being detachably and coaxially connected to said first mating end portion, and bringing said signal terminal to detachably and signally connect with said signal connector, said second locking ring being detachably and threadedly connected to said first mating end portion so as to secure said first mating end portion and said second mating end portion in an end-to-end manner, said insertion tube being configured to be insertable through said insertion end portion from the connecting portion to the tubular body portion of the catheter, said third locking ring having a limiting ring portion sleeved on said second mating end portion and movable toward said insertion end portion, and a third engaging ring portion configured to sleeve threadedly on the connecting portion of the catheter.

19. The endoscope device as claimed in claim 18, wherein said third locking ring is rotatably sleeved on said second mating end portion.

20. The endoscope device as claimed in claim 18, wherein said limiting ring portion of said third locking ring is configured for abutment with and for limiting the connecting portion of the catheter when the connecting portion moves toward said connecting tube.

21. The endoscope device as claimed in claim 18, wherein said insertion tube further has a main tube portion connected between said second mating end portion and said insertion end portion, said second mating end portion having an outer diameter greater than an outer diameter of said main tube portion, and being configured for abutment with and for limiting the connecting portion of the catheter when the connecting portion moves toward said connecting tube.

22. A replaceable insertion tube mechanism that is suitable for mounting on a handgrip mechanism of an endoscope device and that is suitable for a catheter to sleeve thereon, the catheter including a connecting portion and a tubular body portion, said replaceable insertion tube mechanism comprising:
an insertion tube unit including an insertion tube having a second mating end portion and an insertion end portion opposite to each other along a length of said insertion tube, and a third locking ring sleeved on said second mating end portion and movable along the length of said insertion tube, said second mating end portion being configured to detachably and coaxially connect with the handgrip mechanism, said insertion tube being configured to be insertable through said insertion end portion from the connecting portion to the tubular body portion of the catheter, said third locking ring having a limiting ring portion sleeved on said second mating end portion and movable toward said insertion end portion, and a third engaging ring portion configured to sleeve threadedly on the connecting portion of the catheter; and
an image capturing unit mounted on said insertion tube and having an image capture device mounted on said insertion end portion, and a signal terminal signally connected to said image capture device and exposed from said second mating end portion, said image capture device being configured to capture image outside an end of the tubular body portion of the catheter, said signal terminal being configured to signally connect with the handgrip mechanism when said second mating end portion is connected to the handgrip mechanism.

23. The replaceable insertion tube mechanism as claimed in claim 22, wherein said signal terminal is exposed on an end surface of said second mating end portion for detachable electrical connection with the handgrip mechanism.

24. The replaceable insertion tube mechanism as claimed in claim 23, wherein said signal terminal protrudes outwardly from said end surface of said second mating end portion for detachable insertion into the handgrip mechanism.

25. The replaceable insertion tube mechanism as claimed in claim 23, further comprising a first mating tubes extended and inserted into said insertion tube, said first mating tube having a first mating connector exposed on said end surface of said second mating end portion for detachable connection with the handgrip mechanism.

26. The replaceable insertion tube mechanism as claimed in claim 25, wherein said first mating connector protrudes outwardly from said end surface of said second mating end portion for detachable insertion into the handgrip mechanism.

27. The replaceable insertion tube mechanism as claimed in claim 25, further comprising a second mating tube extended and inserted into said insertion tube, said second mating tube having a second mating connector exposed on said end surface of said second mating end portion for detachable connection with the handgrip mechanism.

28. The replaceable insertion tube mechanism as claimed in claim 27, wherein said second mating connector protrudes outwardly from said end surface of said second mating end portion for detachable insertion into the handgrip mechanism.

29. The replaceable insertion tube mechanism as claimed in claim 22, wherein the handgrip mechanism includes a connecting tube, and a first locking ring sleeved over the connecting tube, said second mating end portion having an outer peripheral surface provided with threads for detachable threaded engagement with the first locking ring.

30. The replaceable insertion tube mechanism as claimed in claim 22, wherein said insertion tube unit further includes a second locking ring sleeved over said second mating end portion and movable along the length of said insertion tube for detachable threaded engagement with the handgrip mechanism.

31. The replaceable insertion tube mechanism as claimed in claim 22, wherein said third locking ring is rotatably sleeved on said second mating end portion.

32. The replaceable insertion tube mechanism as claimed in claim 22, wherein said limiting ring portion of said third locking ring is configured for abutment with and for limiting the connecting portion of the catheter when the connecting portion moves toward the handgrip mechanism.

33. The replaceable insertion tube mechanism as claimed in claim 22, wherein said insertion tube further has a main tube portion connected between said second mating end portion and said insertion end portion, said second mating end portion having an outer diameter greater than an outer diameter of said main tube portion and being configured for abutment with and for limiting the connecting portion of the catheter when the connecting portion moves toward the handgrip mechanism.

34. A catheter for mounting on said endoscope device as claimed in claim 1, said catheter including a connecting portion and a tubular body portion integrally connected to each other, wherein:
said connecting portion is configured for said insertion end portion of said insertion tube to be inserted thereinto such that said connecting portion is sleeved on said insertion tube, is movable toward said connecting tube along said insertion tube so as to abut against and be limited by one of said first engaging ring portion and said second mating end portion, and is threadedly engageable with said second engaging ring portion, said tubular body portion being configured to receive said insertion tube when said insertion end portion is inserted into said connecting portion, and having an end opening for allowing said image capture device to capture image.

35. A catheter for mounting on said endoscope device as claimed in claim 10, said catheter including a connecting portion and a tubular body portion integrally connected to each other, wherein:
said connecting portion is configured for said insertion end portion of said insertion tube to be inserted thereinto such that said connecting portion is sleeved on said insertion tube, is movable toward said connecting tube along said insertion tube so as to abut against and be limited by one of said limiting ring portion of said third locking ring (45) and said second mating end portion, and is threadedly engageable with said third engaging ring portion, said tubular body portion being configured to receive said insertion tube when said insertion end portion is inserted into said connecting portion, and having an end opening for allowing said image capture device to capture image.
